(19) United States
(12) Patent Application Publication
(10) Pub. No.: US 2019/0167862 A1
(43) Pub. Date: Jun. 6, 2019

(54) COLLECTION OF AMNIOTIC FLUID FOR WOUND HEALING

(71) Applicant: PENSARA, INC, RICHMOND, TX (US)

(72) Inventor: Geoffrey Charles JONES, Missouri City, TX (US)

(21) Appl. No.: 16/272,245

(22) Filed: Feb. 11, 2019

Related U.S. Application Data
(62) Division of application No. 15/614,550, filed on Jun. 5, 2017.
Application No. 15/614,550 is a continuation-in-part of application No. 14/638,574, filed on Mar. 4, 2015, now Pat. No. 10,029,035.
(60) Provisional application No. 62/353,964, filed on Jun. 23, 2016.

(51) Int. Cl. A61M 1/00; A61B 10/00; A61B 10/02

(52) U.S. Cl. CPC A61M 1/0011; A61M 2202/0494; A61B 10/0283; A61B 10/0048

(57) ABSTRACT

Disclosed are a method of wound healing and an apparatus for the collection, processing and application of amniotic fluid at a wound site to improve healing. The apparatus includes a canister or bag positioned along the vacuum line through which the amniotic fluid and placental aspirate is suctioned. The canister or bag also has a sterile syringe port, through which amniotic fluid can be extracted and later processed. The processed material can then be applied to the wound site of the patient. The apparatus and method disclosed allow for the processing of the amniotic fluid to take place in the same room as the surgical procedure. A kit is provided but not limited to including an amnion rupture tool, a canister or bag for collection, Yankauer suction tip, tubing, and a dual syringe mixing sprayer for reapplication.

### CROSS-REFERENCE TO RELATED U.S. APPLICATIONS

The present application is a division of U.S. application Ser. No. 15/614,550, filed on Jun. 5, 2017, presently pending. U.S. application Ser. No. 15/614,550 is a continuation-in-part of U.S. application Ser. No. 14/638,574, filed on Mar. 4, 2015, which issued as U.S. Pat. No. 10,029,035 on Jul. 24, 2018. U.S. application Ser. No. 15/614,550 also claimed priority to U.S. Provisional Application No. 62/353,964, filed on Jun. 23, 2016.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

The Caesarian Section (C-Section) operation, or the delivery of a baby through the mother's uterus, is one of the most commonly performed procedures in the world. During the late course of a pregnancy, should certain complications arise, a C-Section operation may be necessary to ensure a safe delivery for the mother, the infant, or both. While the C-Section operation is common, the mother is often left with a visible scar from where the primary incision was made. This incision could either be a vertical incision in the middle of the abdomen or a transverse, “bikini-cut” incision made above the pubic hairline. While both incisions can leave a scar, the transverse incision is more commonly performed because it heals to a greater extent than the vertical cut and is less noticeable cosmetically. Additional cuts are made through layers in the abdominal wall to expose the uterine wall. Another incision, usually a horizontal cut, is then made along the uterine wall. During a C-Section, the amniotic fluid in the mother's uterus is simply drained and disposed of, which is actually a waste of valuable, therapeutic biological material, such as stem cells, growth factors, platelets, and enzymes.

The use of stem cells for treatment of wounded tissues is well known. For instance in U.S. Patent Publication No. 2001/09306487 (the '487 publication), published in Apr. 10, 2001 to Grande and Lucas, the use of mesenchymal stem cells in the repair of cartilage tissue is discussed. With the administration of mesenchymal stem cells via a polymeric carrier, the stem cells can differentiate into the same types of cells as the surrounding tissues to promote healing. This publication took quite a narrow view of stem cells in wound healing and did not consider extraction of stem cells from amniotic fluid.

The collection and reuse of amniotic fluid has been contemplated in certain prior applications such as the U.S. Patent Publication No. 2014/0336600 (the '600 publication), published on -3- Nov. 13, 2014 to Harrell. The '600 publication describes a method for obtaining sterile amniotic fluid, as well as uses for such fluid. The '600 publication describes the use of a suction or a pump for collecting the amniotic fluid, as well as separation of cells such as growth factors and stem cells from the amniotic fluid. The '600 publication describes the separation of the growth factors and stem cells in a centrifuge remote from the collection site. The '600 publication, however, fails to consider the addition of a suitable hemostat such that the therapeutic components of the amniotic fluid can adhere and coagulate on the mother's scar during C-Section.

In addition, U.S. Patent Application No. '3097, submitted by the same individual that now submits this patent application, claims a device for the collection and immediate centrifugation of amniotic fluid for processing and autologous administration. The publication describes an apparatus that both collects and centrifuges the amniotic fluid and then allows it to be withdrawn and processed with a suitable hemostat before autologous administration. This device, however, is not capable of actually performing according to its design due to the helical tube within the apparatus, which obstructs the extraction of the amniotic fluid, drastically reducing the viability of the design.

Thus, it is necessary for a system and method wherein stem cells and other important biological material from amniotic fluid can be processed by the addition of a hemostat solution and then administered on the same patient to assist in healing the wound from a C-Section.

It is the object of this invention to provide a system and method to accomplish this goal.

It is another object of this invention to allow for easy collection, processing, and extraction of amniotic fluid with existing operating room equipment.

It is another object of this invention to use this apparatus to promote healing of a C-Section wound.

It is another object of this invention to provide a kit including the components described above.

### BRIEF SUMMARY OF THE INVENTION

One embodiment of the present invention is a method for wound healing. The collection of amniotic fluid at birth, processing of the collected fluid, and its administration to a wound site can improve the quality, the speed, and the extent to which the wound heals. In one embodiment of the present invention, amniotic fluid is collected from the uterus during a C-section operation. The processing of the fluid can include the addition of a suitable hemostat such as calcium chloride or bovine thrombin in order to improve the consistency of the resultant solution. Processing may also include a means to improve the density of the therapeutic biological material such as stem cells, growth factors, and hormones. The processed solution can then be administered to the human or non-human patient at the wound site such that the stem cells may adhere to the wounded tissue. Methods for administration include both dermal injection and spray.

The present invention is also an environment suitable for the differentiation, development, and proliferation of stem cells within the amniotic fluid. Such an environment is usually the product of stressed biological tissues, including tissues where incisions have been made in an operation. These stressed tissues release several important signaling molecules such as growth factors, transcription factors, and growth hormones which promote the differentiation of stem cells into mature cells. This claimed environment is crucial to the appropriate development of new tissues over a wound site.

In another embodiment of the present invention, the invention is an apparatus for the collection and processing of cellular material from amniotic fluid. The apparatus includes a canister having an inlet and an outlet. The inlet is connected to a suction tube and the outlet connected to the vacuum line. A port is provided which is in communication with the chamber. Preferably, the port is positioned adjacent the inlet of the canister. In the present invention, the port may be suitable for receipt of a needle of a syringe therein.

The present invention is also a kit for the collection, separation and use of cellular material from amniotic fluid. The kit has a canister similar to that of a mucus trap or the canister could be a modified mucus trap. The canister has a syringe port along with an inlet and an outlet. The canister is suitable for attachment to a vacuum line to the outlet port. The kit also contains a syringe having a needle suitable for insertion into the port of the cellular accumulation chamber. Finally, a dual chambered applicator is provided which can be used to mix the extracted amniotic fluid with a coagulant or hemostat. The kit may additionally contain a Yankauer dental tip for the collection of amniotic fluid. This tip may contain a plurality of teeth for scraping the collection site.

Importantly, in the method for collection of amniotic fluid within the present invention, each of the steps is conducted in a single operating room or laboratory coincident with the surgical procedure.

### BRIEF DESCRIPTION OF THE SEVERAL VIEWS OF THE DRAWINGS

FIG. 1 illustrates a basic outline of the method of the present invention.

FIG. 2 shows a perspective and an exploded view of one embodiment of the present invention.

FIG. 3 shows a perspective view of one embodiment of the apparatus of the present invention.

FIG. 4 shows an exploded view of one embodiment of the kit of the present invention.

### DETAILED DESCRIPTION OF THE INVENTION

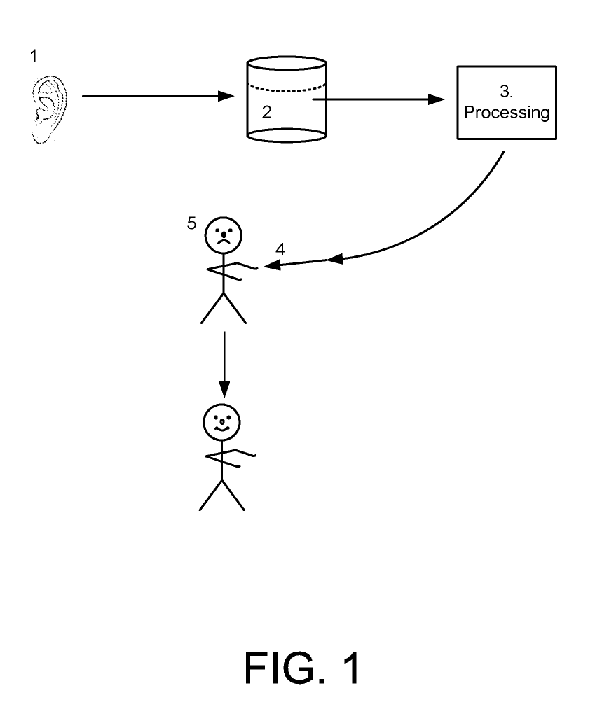

FIG. 1 shows a basic outline of the method claimed. Extracted amniotic fluid (1) is collected in a sterile container (2). The sterile container prevents contamination of the solution during the course of the coincident operation, which may be a C-Section operation. After collection, amniotic fluid can be withdrawn from the container and processed (3). Once the solution has an appropriate density and consistency, it can be administered (4) to the wound of a patient (5). Because of the therapeutic capability of the solution, the wound healing is fast, clean, and effective (6).

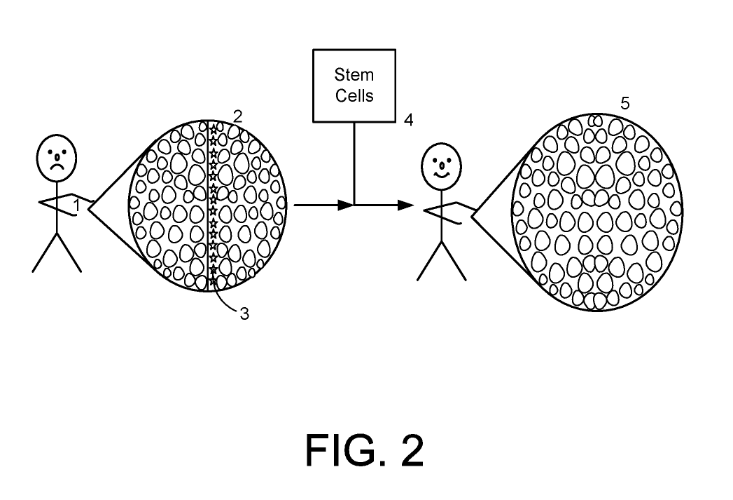

FIG. 2 shows a perspective and an exploded view of one embodiment of the environment claimed. In this embodiment, an incision (1) is clearly visible on the skin of a patient. A blown up view (2) of the stressed tissue would indicate the propagation of a growth factor cascade, the release of several chemical signals (3). As the stem cells are applied to the wound (4), these signaling molecules promote the differentiation and proliferation of these cells (5).

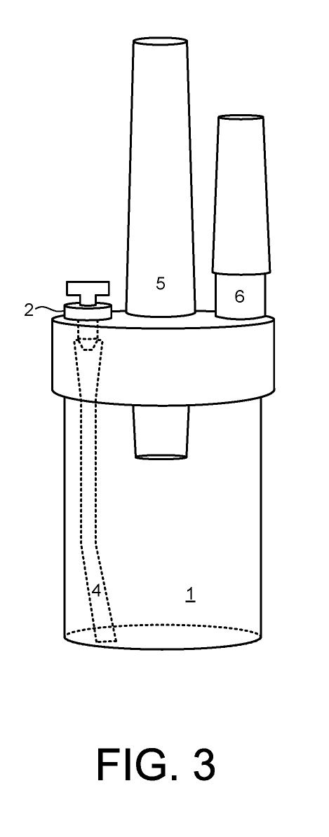

FIG. 3 shows a perspective view of one embodiment of the apparatus claimed. The apparatus of FIG. 3 implements a modified mucus trap with an additional sterile syringe port. In this embodiment, the canister (1) is capable of storing approximately 800 cubic centimeters of amniotic fluid. The sterile syringe port (2) consists of a female luer lock (3) spanning across the top membrane of the mucus trap. The luer lock fits snugly against the membrane of the mucus trap such that the inner canister of the mucus trap is sealed. The portion of the luer lock within the canister connects to a rubber hose (4) that extends down a few centimeters from the bottom of the mucus trap. The female portion of the luer lock, which faces the outside of the canister, is suited for a syringe to lock into for the withdrawal of amniotic fluid after collection. In addition, the two ports characteristic to a mucus trap are utilized as an inlet port (5) and an outlet port (6) respectively. When a hose is placed to connect the outlet port to a medical vacuum pump, the outlet port creates a vacuum line and evolves a lower pressure within the canister. A hose can be connected to the inlet port and can be placed within the uterus of the birthing mother to collect the amniotic fluid with a force caused by the differential in pressure between the inside of the canister and the surrounding atmosphere. Because the inside of the canister has a lower pressure, the pressure from the surrounding atmosphere forces the amniotic fluid through the hose and the inlet port and into the canister.

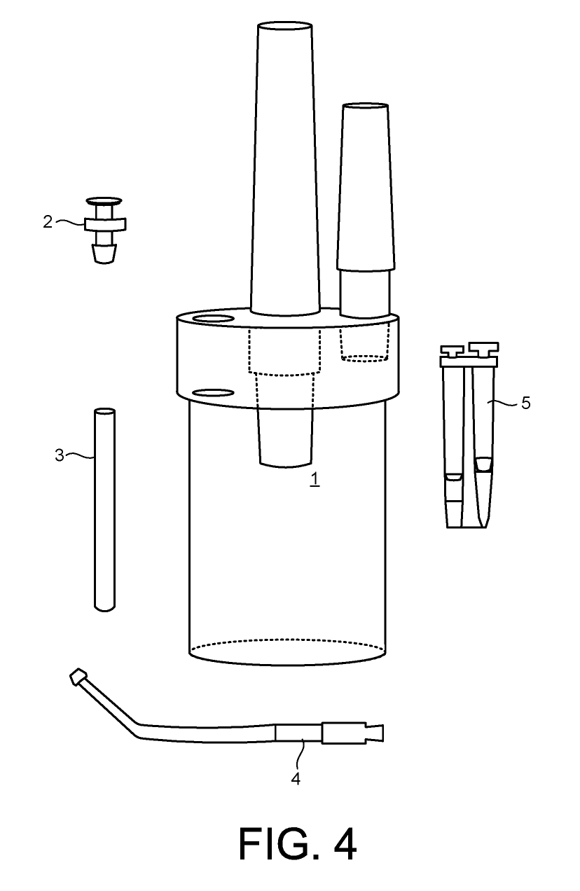

FIG. 4 shows an exploded view of one embodiment of the kit claimed. In FIG. 4, the device shown in FIG. 3 is shown in an exploded view such that the mucus trap (1) with an additional hole for the syringe port is shown apart from the female luer lock (2) and the rubber hose (3). In addition, this embodiment of the kit includes a Yankauer tip (4) that can be placed on the inlet hose to aid in the collection of amniotic fluid from the mother's uterus. The Yankauer tip may have teeth that aid in the collection of biological material. This embodiment of the kit also includes a dual chambered applicator (5) while a suitable coagulant or hemostat can occupy space in the other chamber such that the two can mix before administration.

In one embodiment of the apparatus or kit, the mixture of amniotic fluid and a coagulant or hemostat can then feed into a sprayer device such that the therapeutic biological material from the amniotic fluid can coagulate on the wound from the Caesarian section.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. A method for healing wounds comprising:
use of amniotic fluid during a medical procedure;
processing of said amniotic fluid to improve its efficacy for the purposes of wound healing; and
administration of said amniotic fluid on a wound of interest, whereby said wound may be healed.

2. The method of claim 1, wherein said medical procedure is either a Caesarian section operation or a natural child birth.

3. The method of claim 1, wherein said processing includes the addition of a hemostat.

4. The method of claim 3, wherein said hemostat is selected from a group consisting of: a calcium chloride solution and a thrombin solution.

5. The method of claim 1, wherein said administration includes the use of a sprayer whereby therapeutic material may be thinly and evenly applied over the surface of the wound.

6. The method of claim 5, wherein said sprayer is a dual syringe including amniotic fluid and hemostat.

7. The method of claim 1, wherein said administration includes dermal injection.

8. The method of claim 1, wherein said administration is autologous or non-autologous.

9. The method of claim 8, wherein the non-autologous administration is the administration of therapeutic amniotic fluid on the fetus of the birthing mother.

10. The method of claim 8, wherein the non-autologous administration is on a human patient.

11. The method of claim 8, wherein the non-autologous administration is on a non-human patient.

12. The method of claim 1, wherein said processing includes cryogenic preservation of said amniotic fluid for future use.

13. The method of claim 12, wherein the future use includes autologous administration of said amniotic fluid on the original donor and human patient to heal the wounds of said patient.

14. The method of claim 12, wherein the future use includes non-autologous administration of said amniotic fluid on a separate human patient to heal the wounds of said separate human patient.

15. The method of claim 1, wherein said amniotic fluid is extracted during an amniocentesis procedure.

16. The method of claim 1, wherein said environment includes additional growth factors added to the solution.

17. The method of claim 1, further comprising:
collecting said amniotic fluid in a mucus trap.

18. The method of claim 1, said step of processing comprising:
use of a dual syringe to create a solution capable of improving wound healing.
